(19) United States
(12) Patent Application Publication
(10) Pub. No.: US 2024/0390658 A1
(43) Pub. Date: Nov. 28, 2024

(54) MEDICAL SYSTEMS, DEVICES, AND METHODS FOR DELIVERING ONE OR MORE FLUIDS

(71) Applicants: Boston Scientific Scimed, Inc., Maple Grove, MN (US); Boston Scientific Medical Device Limited, Galway (IE)

(72) Inventors: Deepak Kumar SHARMA, Muzaffarnagar (IN); Kevin L. BAGLEY, Natick, MA (US); Sharath Kumar G, Kanakapura (IN); Mazen YATIM, Southborough, MA (US); Samuel GOSHEN, Brookline, MA (US)

(73) Assignees: Boston Scientific Scimed, Inc., Maple Grove, MN (US); Boston Scientific Medical Device Limited, Galway (IE)

(21) Appl. No.: 18/669,697

(22) Filed: May 21, 2024

Related U.S. Application Data
(60) Provisional application No. 63/504,275, filed on May 25, 2023.

(51) Int. Cl. A61M 31/00

(52) U.S. Cl. CPC A61M 31/00; A61M 2202/0007; A61M 2205/582; A61M 2205/583; A61M 2210/105; A61M 2210/1053; A61M 2210/106; A61M 2210/1064

(57) ABSTRACT

A medical device may include a handle and an end cap. The handle may include a first movable body and a stationary body. The end cap may include an outer portion, an inner portion disposed within the outer portion, and at least one biasing member. The outer portion may define a receptacle configured to contain a material. The inner portion may be proximal to the receptacle, within the outer portion, and coupled to the first movable body by at least one control element. The biasing member(s) may be disposed between a proximal face of the inner portion and a distal face of a proximal wall of the outer portion. Accordingly, movement of the first movable body relative to the stationary body moves the inner portion relative to the outer portion. Movement of the inner portion may be configured to deliver the material.

### CROSS-REFERENCE TO RELATED APPLICATIONS

This application claims the benefit of priority under 35 U.S.C. § 119 from U.S. Provisional Application No. 63/504,275, filed May 25, 2023, which is incorporated by reference herein in its entirety.

### TECHNICAL FIELD

This disclosure generally relates to medical systems, devices, and related methods that may be used to treat a subject. More particularly, at least some embodiments of the present disclosure relate to systems, devices, and related methods for delivering one or more fluids.

### BACKGROUND

In certain medical procedures, it may be necessary to remove a portion of a tissue and/or to stop or minimize bleeding internal to the body. For example, some endoscopic medical procedures may require removing tissue (e.g., polyps, lesions, etc.) within the gastrointestinal tract, for example, in the esophagus, stomach, or intestines. Other procedures may require hemostasis of bleeding tissue within the gastrointestinal tract. Physicians have adopted minimally invasive techniques for removing tissue, such as endoscopic procedures like endoscopic mucosal resection (EMR) and endoscopic submucosal dissection (ESD). EMR methods are typically used for removal of small cancerous or abnormal tissues (e.g., polyps), and ESD methods are typically used for en bloc removal of large cancerous or abnormal tissues (e.g., lesions). Physicians have also adopted minimally invasive techniques to stop bleeding internal to the body.

In these procedures, materials (e.g., fluids, substances, powders, and/or agents) may be delivered to a treatment site within the gastrointestinal tract using systems or devices. Such systems or devices, however, may require medical professionals to exert or apply great amounts of force to deliver the material to the treatment site. Accordingly, medical professionals may experience fatigue and/or discomfort from exerting large amounts of force over the course of a procedure. Over time, for example, medical professionals may experience symptoms similar to those of Carpal Tunnel Syndrome, tendonitis, or De Quervain's tenosynovitis. When a medical professional experiences fatigue or other pain in the fingers, hand, or wrist, the medical professional may take breaks from the procedure and/or readjust his or her hand position. When a medical professional takes breaks and/or repeatedly readjusts his or her handgrip in between procedure tasks, the procedure may be prolonged and procedural tasks may be more difficult.

Furthermore, the application of the aforementioned forces may pose a risk of various components of the systems and/or devices breaking or otherwise malfunctioning over the course of the procedure. If the systems and/or devices break or malfunction, for example, within a subject, the subject may have an increased risk of harm or injury. For example, the broken systems and/or devices may perforate, cut, puncture, or otherwise injure the subject's (e.g., the subject's gastrointestinal tract). Alternatively or additionally, the medical professional may have to delay or otherwise extend the duration of the procedure to retrieve, repair, or otherwise address the broken component.

The systems, devices, and methods of this disclosure may rectify some of the deficiencies described above or address other aspects of the art.

### SUMMARY

Examples of this disclosure relate to, among other things, systems, devices, and methods for performing one or more medical procedures. Specifically, this disclosure includes medical systems and devices comprising an end effector that is configured to deliver a material and methods of use thereof (e.g., methods of delivering the end effector to a treatment site of a subject, for example, to deliver the material). Each of the examples disclosed herein may include one or more of the features described in connection with any of the other disclosed examples.

According to one aspect, a medical device may include a handle and an end cap. The handle may include a first movable body and a stationary body. The end cap may include an outer portion, an inner portion, and at least one biasing member. The inner portion may be disposed within the outer portion. The outer portion of the end cap may define a receptacle configured to contain a material. The inner portion may be proximal to the receptacle within the outer portion. The inner portion may be coupled to the first movable body by at least one control element. The at least one biasing member may be disposed between a proximal face of the inner portion and a distal face of a proximal wall of the outer portion. Accordingly, movement of the first movable body relative to the stationary body may move the inner portion of the end cap relative to the outer portion of the end cap. Movement of the inner portion of the end cap may be configured to deliver the material.

In a first configuration, the at least one control element may have a first tension, the at least one biasing member may have a first length, and the inner portion may be in a first position. In a second configuration, the at least one biasing member may have a second length greater than the first length, the at least one biasing member may have a second length greater than the first length, and the inner portion may be in a second position. In the first configuration, the receptacle may have a first volume. In the second configuration, the receptacle may have a second volume. The second volume may be less than the first volume.

In some embodiments, the end cap may further include a nozzle and at least one tubular member extending distally from a distalmost face of the outer portion. The at least one tubular member may be fluidly connected to the nozzle and the outer portion. The material may be delivered via the at least one tubular member and the nozzle. In some embodiments, the nozzle may include a plurality of internal threads.

The receptacle may include at least a first section and a second section. At least the first section and the second section may be partitioned by a wall. The first section may be configured to contain the first material. The second section may be configured to contain a second material. The first section of the receptacle may have a first volume, and the second section of the receptacle may have a second volume different from the first volume.

In some embodiments, the end cap may include at least one port arranged on an external surface of the outer portion. The at least one portion may include a one-way valve. The at least one control element may be movable disposed within a lumen of a sheath. The sheath may be coupled to the outer portion of the end cap. The end cap may be removably coupled to an endoscope by one or more fasteners, adhesives, or a friction fit. The end cap may include an aperture extending from a proximal end to a distal end. The aperture may be configured to receive a distal portion of the scope.

In some embodiments, the inner portion may include a proximal portion and a distal portion. The proximal portion may be a rigid material. The distal portion may be a flexible material. The distal portion of the inner portion may form a liquid-tight seal with the outer portion of the end cap. The handle may include at least one indicator. The indicator may indicate a volume of the material disposed within the receptacle of the end cap.

According to another aspect of this disclosure, a medical device may include an end cap. The end cap may include a fixed portion, a first movable portion, and a second movable portion. The fixed portion of the end cap may include a first section and a second section partitioned by a wall. The first section and the second section may be configured to receive first and second materials, respectively. The first movable portion may be disposed within the first section, and the second movable portion may disposed within the second section. In a first configuration, the first movable portion may be in a proximal position within the first section of the fixed portion. The second movable portion may be in a proximal position within the second section of the fixed portion. Each of the first portion and the second portion may have a first volume. In a second configuration, the first movable portion may be in a distal portion within the first section of the fixed portion such that the first section has a second volume. The second volume may be less than the first volume.

In the second configuration of some embodiments, the second movable portion may be in a distal position within the second section of the fixed portion such that the second section has a second volume. The second volume may be less than the first volume.

In some embodiments, the end cap may include at least two biasing members. A first of the at least two biasing members may be disposed between a proximal face of the first movable portion and a distal face of a proximal wall of the fixed portion. A second of the at least two biasing members may be disposed between a proximal face of the second movable portion and the distal face of the proximal wall of the fixed portion. In the first configuration, each of the at least two biasing members may have a first length. In the second configuration, the first of the at least two biasing members may have a second length. The second length may be greater than the first length.

Aspects of this disclosure further include a method. The method may include delivering a medical system to a treatment site. The medical system may include a scope, a medical device, and a material. The medical device may include a handle having a first movable body and a stationary body. The end cap may be removably coupled to a distal end of the scope. The end cap may include an inner portion, an outer portion, and at least one biasing member. The inner portion may be coupled to the first movable body of the handle by one or more control elements. The outer portion may define a receptacle. The at least one biasing member may be disposed between a proximal face of the inner portion and a distal face of a proximal wall of the outer portion. The material may be disposed within the receptacle of the end cap.

The method may further include moving the first movable body relative to the stationary body by a first distance such that a tension in the one or more control elements is decreased by a first amount and the at least one biasing member is extended by a first amount to move the inner portion of the end cap relative to the outer portion of the end cap by a first distance. The method may also include delivering a first amount of the material.

The method may further include repositioning the medical system relative to the treatment site and moving the first movable body relative to the stationary body by a second distance such that a tension a tension in the one or more control elements is decreased by a second amount and the at least one biasing member is extended by a second amount to move the inner portion of the end cap relative to the outer portion of the end cap by a second distance. The method may also include delivering a second amount of the material.

Any of the examples described herein may have any of these features in any combination.

### BRIEF DESCRIPTION OF THE DRAWINGS

The accompanying drawings, which are incorporated in and constitute a part of this specification, illustrate exemplary aspects of the disclosure and together with the description, serve to explain the principles of the disclosure.

FIG. 1A illustrates a perspective view of an exemplary medical system, including an exemplary medical device and a portion of an endoscope, according to aspects of this disclosure.

FIG. 1B illustrates a perspective view of a distal portion of the medical device of FIG. 1A coupled to a distal portion of the endoscope, according to aspects of this disclosure.

FIG. 2 is a flow diagram of an exemplary method, according to aspects of this disclosure.

### DETAILED DESCRIPTION

Reference will now be made in detail to aspects of the present disclosure, examples of which are illustrated in the accompanying drawings. Wherever possible, the same or similar reference numbers will be used through the drawings to refer to the same or like parts. The term “distal” refers to a portion farthest away from a user when introducing a device into a subject (e.g., subject). By contrast, the term “proximal” refers to a portion closest to the user when placing the device into the subject. Proximal and distal directions are labeled with arrows marked “P” and “D”, respectively, throughout various figures.

Both the foregoing general description and the following detailed description are exemplary and explanatory only and are not restrictive of the features, as claimed. As used herein, the terms “comprises,” “comprising,” “having,” “including,” or other variations thereof, are intended to cover a non-exclusive inclusion such that a process, method, article, or apparatus that comprises a list of elements does not include only those elements, but may include other elements not expressly listed or inherent to such a process, method, article, or apparatus. In this disclosure, relative terms, such as, for example, “about,” “substantially,” “generally,” and “approximately” are used to indicate a possible variation of ±10% in a stated value or characteristic.

Although the treatment site is discussed herein as being in the subject's gastrointestinal tract, this disclosure is not so limited, as the treatment site may be any internal lumen, organ, cavity, or other tissue within the subject. Additionally, although endoscopes are referenced herein, it will be appreciated that the disclosure encompasses any medical devices having a working channel, or a lumen, extending from a proximal end to a distal end, such as ureteroscopes, duodenoscopes, gastroscopes, endoscopic ultrasonography (“EUS”) scopes, colonoscopes, bronchoscopes, laparoscopes, arthroscopes, cystoscopes, aspiration scopes, sheaths, or catheters.

Embodiments of the disclosure may address one or more of the limitations in the art. The scope of the disclosure, however, is defined by the attached claims and not the ability to solve a specific problem. The present disclosure is drawn to systems, devices, and related methods, for a tissue lifting medical device, among other aspects.

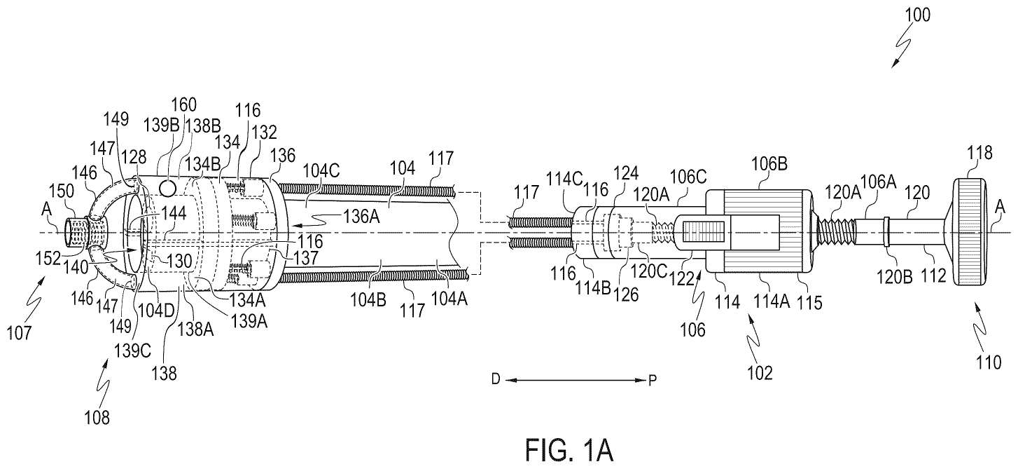

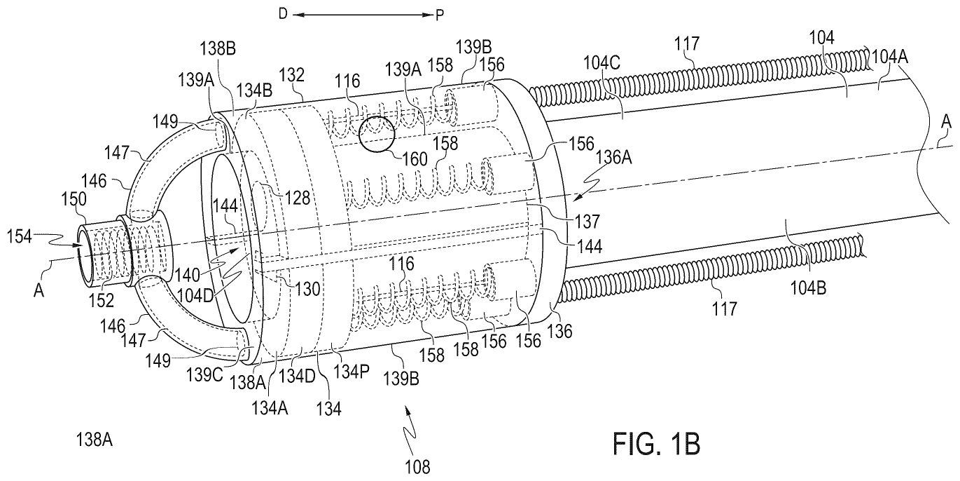

FIG. 1A depicts a perspective view of a medical system 100, for example, including a medical device 102 and an endoscope 104. As discussed in detail herein, medical device 102 may include a handle 106 and an end cap 108. End cap 108 may comprise a distal end 107 of medical device 102. End cap 108 may be coupled to handle 106 via one or more control elements 116 and/or one or more sheath elements 117.

In some embodiments, end cap 108 of medical device 102 may be coupled to a distal portion 104C (e.g., a distal end) of endoscope 104. End cap 108 may be coupled to endoscope 104 before insertion of endoscope 104 into the subject, such that end cap 108 and distal portion 104C of endoscope 104 may be delivered to the treatment site together. Then, the user may manipulate one or more portions of handle 106 to control (e.g., extend or retract) one or more portions of end cap 108 to deliver a material (e.g., a fluid, a liquid, a gas, a substance, etc.) contained within a portion of end cap 108. Furthermore, although not shown, endoscope 104 may include a handle with one or more proximal controls (knobs, dials, levers, buttons, etc.), for example, to extend or retract, deflect, articulate, or otherwise control the position of distal portion 104C of endoscope 104, and thus control the position of end cap 108 before, during, and/or after delivering the material.

As mentioned, a proximal end 110 of medical device 102 may include handle 106. Handle 106 may include a proximal handle portion 106A, an intermediate handle portion 106B, and a distal handle portion 106C. Proximal handle portion 106A may include a first movable body 112. Intermediate handle portion 106B and/or distal handle portion 106C may include a stationary body 114. For example, intermediate handle portion 106B may include a first portion 114A of stationary body 114 and distal handle portion 106C may include a second portion 114B of stationary body 114.

Specifically, first movable body 112 may be positioned on a proximal end of proximal handle portion 106A. First movable body 112 may include one or more knobs, dials, sliders, levers, buttons, etc. Movement of first movable body 112 relative to stationary body 114 may control the movement (e.g., extension or retraction) of one or more control elements 116, for example, control wire(s). As will be discussed further herein, control element(s) 116 may control the movement (e.g., extension or retraction) of one or more portions of end cap 108. In these aspects, at least a portion of first movable body 112 may be coupled (e.g., directly or indirectly) to or otherwise connected to control element(s) 116.

In some embodiments, first movable body 112 includes a knob 118 and a shaft 120. In some aspects, knob 118 is fixed to or otherwise securely coupled to a proximal end of shaft 120. Knob 118 may include one or more ridges or indentations, for example, to facilitate a user's grip on first movable body 112. At least a portion of first movable body 112 (e.g., shaft 120) may extend into and/or through first portion 114A and/or second portion 114B of stationary body 114. For example, in some embodiments, a portion of shaft 120 may extend into first portion 114A of stationary body 114. In other embodiments, a portion of shaft 120 may extend through first portion 114A of stationary body 114 and at least partly into second portion 114B of stationary body 114. Although the terms “movable” and “stationary” are used herein, it is noted that the relative movement may be reversed. For example, stationary body 114 may be rotated relative to first movable body 112.

Rotation of knob 118 about an axis A results in rotational movement of shaft 120 and, thus, extension and/or retraction of control element(s) 116. Axis A is a center axis of medical device 102 that extends from handle 106 to end cap 108. Although not shown, one or more internal features of first portion 114A and/or second portion 114B of stationary body 114 is/are configured to engage with a plurality of threads 120A extending about at least a portion of shaft 120. Threads 120A may extend spirally around at least the portion of a radially-outer portion of shaft 120, for example, forming a threaded portion of shaft 120. For example, first portion 114A and/or second portion 114B may include one or more complementary internal threads configured to engage with plurality of threads 120A on shaft 120. In such a way, axial movement of first moveable body 112 may be controlled. For example, rotation of first movable body 112 in a first rotational direction (e.g., clockwise or counterclockwise) may result in axial movement of first movable body 112 in a first axial direction (e.g., distally or proximally) relative to stationary body 114. Rotation of movable body 112 in a second rotational direction opposite to the first rotational direction (e.g., counterclockwise or clockwise) may result in axial movement of first movable body 112 in a second axial direction opposite to the (e.g., proximally or distally) relative to stationary body 114.

In some embodiments, a projection or rib 120B extends at least partially around a portion of shaft 120, for example, proximal of plurality of threads 120A. Rib 120B may be configured to help prevent axial movement of first movable body 112 beyond rib 120B. For example, rib 120B may be configured to engage with or abut a proximal portion (e.g., a proximalmost face) of first portion 114A of stationary body 114, for example, to help prevent axial movement of first movable body 112 beyond rib 120B.

First portion 114A of stationary body 114 may include a gripping surface 115, which may include one or more ridges and/or indentations, for example, to facilitate a user's grip on stationary body 114. A cross section of first portion 114A may be substantially circular, ovular, square, rectangular, or any other shape. In some embodiments, first portion 114A is formed similar to a barrel or a rigid tube. First portion 114A may be hollow and configured to receive a portion of first movable body 112 (e.g., shaft 120). For example, an inner diameter of first portion 114A may be complimentarily sized and shaped to receive a portion of shaft 120 and permit rotational and/or axial movement of shaft 120 within first portion 114A, as discussed above.

First portion 114A of stationary body 114 may further include a second movable body 122. Second movable body 122 may be a knob, a switch, a lever, a button, or any similar mechanism commonly used in the art. In a first position (e.g., a neutral position), second movable body 122 may be configured to permit rotational movement of first movable body 112 within first portion 114A of stationary body 114, as previously discussed. Additionally or alternatively, second movable body 122 may be configured as a braking mechanism for first moveable body 112. For example, at least a portion of second movable body 122 may be moved (e.g., towards a central axis of stationary body 114) such that movable body 122 presses against first movable body 112, for example, within first portion 114A of stationary body 114. In such a way, second movable body 122 may be configured to help inhibit, or prevent, rotational and/or axial movement of first movable body 112. In such a way, second movable body 122 may be configured to fix first movable body 112 in a desired position.

Additionally or alternatively, second movable body 122 may be configured as a “quick-release” mechanism for first movable body 112. For example, in a third position (e.g., an unlocked or open position), second movable body 122 may be configured to disengage from or release first movable body 112 such that first movable body 112 may be rotated and/or moved in an axial direction (e.g., along distally or proximally) more quickly and/or easily. In such a way, first movable body 112 may be rotated or moved in an axial direction to actuate (e.g., extend or retract) one or more portions of distal cap 108 quickly. In some aspects, although not shown, second movable body 122 may be biased in the first, second, or third position by one or more biasing elements (e.g., springs, hinges, etc.). In some configurations, the first, second, and third positions of movable body 122 are longitudinally spaced. For example, the second position may be distal to the first position, and the third position may be proximal to the first position.

Referring still to FIG. 1A, first portion 114A and second portion 114B of stationary body 114 may be a single, unitary structure formed from a single material (e.g., formed by molding, three-dimensional printing, etc.). Alternatively, first portion 114A and second portion 114B may be separate components coupled via one or more mechanical fasteners (e.g., screws, press-fits, welds, etc.), a friction fit, and/or chemical fasteners (e.g., glues, adhesives, resins, etc.). Similar to first portion 114A, a cross section of second portion 114B may be circular, ovular, square, rectangular, or any other shape. In some embodiments, and as shown in FIG. 1A, second portion 114B may have a smaller diameter as compared to first portion 114A.

Additionally or alternatively, second portion 114B may have a same cross sectional shape and/or size as first portion 114A or a different cross sectional shape and/or size. For example, first portion 114A and second portion 114B may both have a circular cross section. Alternatively, first portion 114A may have a rectangular cross section, and second portion 114B may have a circular cross section. In some embodiments, second portion 114B may be formed similar to a barrel or a rigid tube. Second portion 114B may be hollow and configured to receive a distal portion of first movable body 112, such as, for example, a distal portion 120C of shaft 120. Distal portion 120C may be distal to plurality of threads 120A of shaft 120. For example, an inner diameter of second portion 114B may be complimentarily sized and/or shaped to receive distal portion 120C of shaft 120. Second portion 114B may be configured to permit rotational and/or axial movement of first movable body 112 within second portion 114B.

Second portion 114B may include one or more visual or physical indicator(s) 124. Indicator(s) 124 may include, for example, markings, indentations, raised surfaces, etc. Indicator(s) 124 may visually or physically indicate the actuation or extension of end cap 108. Second portion 114B may be made from a clear, transparent, or semi-transparent material to permit visualization of first movable body (e.g., distal portion 120C of shaft 120) within second portion 114B. For example, as first movable body 112 is advanced distally relative to stationary body 114, shaft 120 is advanced distally within second portion 114B. Indicator(s) 124 may signal to the user that end cap 108 has been moved or actuated, and/or that a fluid or a material within end cap 108 has been at least partially delivered. Additionally or alternatively, the user may utilize markings on handle 106 or other visual indicators to determine a status, position, actuation status, etc. of one or more portions of end cap 108.

Control element(s) 116 extend proximally into second portion 114B, for example, through respective openings (not shown) in a distalmost face 114C of second portion 114B. As previously discussed, a proximal portion of control element(s) 116 may be coupled (e.g., directly or indirectly) to first movable body 112. For example, control element(s) 116 may be coupled to distal portion 120C of shaft 120. In some embodiments, control element(s) 116 may be coupled to a distal portion of a socket 126. A proximal portion of socket 126 may be configured to receive distal portion 120C of shaft 120. In such a way, shaft 120 may be movably coupled to socket 126 such that, when shaft 120 is rotated within socket 126, socket 126 does not rotate. Accordingly, control element(s) 116 is/are not twisted or rotated as shaft 120 is rotated. Further, when shaft 120 is moved longitudinally (e.g., proximally and/or distally), socket 126 moves correspondingly with shaft 120. For example, as shaft 120 rotates in a first direction and moves distally, socket 126 moves distally, but does not rotate. Alternatively, as shaft 120 rotates in a first direction and moves proximally, socket 126 moves proximally, but does not rotate.

A distal portion of control element(s) 116 may be coupled (e.g., directly or indirectly) to end cap 108 such that, as first movable body 112 (including shaft 120) is rotated within socket 126 and axially moved within second portion 114B, control element(s) 116 are axially translated (e.g. extended and/or retracted). The axial translation of control element(s) 116 may correspondingly move (e.g., extend and/or retract) a portion of end cap 108.

Although not shown, medical device 102 may include two or more handles 106. For example, each of the two or more handles 106 may be configured to control the movement or position of one or more control element(s) 116. In such a way, control element(s) 116 coupled to each handle 106 may be independently controlled, for example, to control different portions of end cap 108. In further alternative embodiments, medical device 102 may include two or more first movable bodies 112 on or otherwise coupled to handle 106. For example, handle 106 may include two or more first movable bodies 112 configured to control one or more control element(s) 116 such that control element(s) 116 coupled to each of the two or more first movable bodies 112 may be independently controlled.

Still referring to FIG. 1A, control element(s) 116 may each be formed by a wire, a cable, a coil, a tube, etc. Each control element 116 may be radially positioned and moveable within a respective sheath element 117 (e.g., control element 116 may be moveably disposed within a lumen of sheath element 117). Accordingly, in these aspects, sheath element(s) 117 does/do not move with the movement of control element(s) 116. Each sheath element 117 may extend from one or more portions of handle 106 (e.g., distal face 114C) to one or more portions of end cap 108. Sheath element(s) 117 may each be formed by a coil, a tube, a sheath, etc., and may have an internal lumen (not shown) extending longitudinally to movably receive a respective control element(s) 116.

Endoscope 104 may include a generally cylindrical tubular shape, and may include a proximal portion 104A, an intermediate portion 104B, and distal portion 104C. Although not shown, proximal portion 104A may include or otherwise be coupled to a handle, for example, including one or more ports, controls, levers, buttons, electrical communication connections, etc. Additionally, endoscope 104 may include one or more working channels or lumens 128. Lumen(s) 128 may extending longitudinally through endoscope 104, for example, extending through proximal portion 104A, intermediate portion 104B, and distal portion 104C. Although not show, lumen(s) 128 may extend to or otherwise be connected to one or more ports on the handle.

Endoscope 104 (i.e., distal portion 104C of endoscope 104) may include an outer diameter of approximately 9 millimeters to approximately 20 millimeters, for example, approximately 10.5 millimeters to approximately 12 millimeters. As mentioned, endoscope 104 may include lumen(s) 128, for example, a working channel with a diameter of approximately 2 millimeters to approximately 4 millimeters, for example, approximately 2.8 millimeters. One or more lumen(s) 128 may terminate at a distal face 104D of endoscope 104. Additionally, distal face 104D of endoscope 104 may include one or more devices 130. Device(s) 130 may include one or more visualization devices (e.g., cameras, image sensors, lenses, etc.), one or more illumination devices (e.g., LEDs, optical fibers, etc.), treatment devices (e.g., laser fibers, elevators, etc.), or one or more other devices to image, view, or otherwise treat a treatment site.

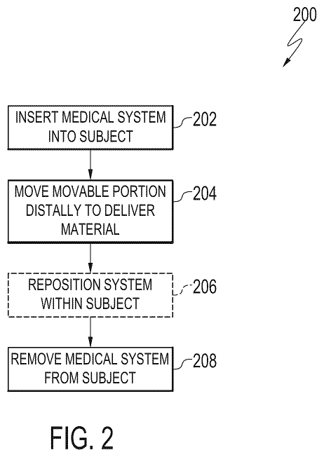

As shown in FIG. 1A and FIG. 2, when medical device 102, including end cap 108, is coupled to endoscope 104 (i.e., during delivery and positioning of endoscope 104), end cap 108 and/or a distal portion of medical device 102 may be coupled to distal portion 104C of endoscope 104. For example, distal portion of medical device 102 and/or a proximal portion of end cap 108 may be coupled to an external surface of distal portion 104C of endoscope 104.

The proximal portion of end cap 108 may include an internal diameter that is complementary to and may receive an exterior diameter of endoscope 104. End cap 108 may be attached to distal portion 104C of endoscope 104 by methods of, for example, an adhesive, a friction fit, a mechanical fit, a snap fit, a press fit, a weld, or any other coupling techniques commonly known in the art. Additionally or alternatively, end cap 108 may be coupled to and/or extend distally beyond the distal face 104D. By coupling end cap 108 to an external surface of distal portion 104C of endoscope 104, the size and/or shape of end cap 108 may not be limited by the size and/or shape of lumen(s) 128. Furthermore, in this configuration, lumen(s) 128 may be larger or endoscope 104 may have additional lumens for the insertion of additional medical instruments, the delivery of additional materials, the application of suction, etc. Additionally or alternatively, endoscope 104 may have a smaller overall diameter.

Although not shown, a portion of end cap 108 may extend proximally into one or more lumens 128 and couple to an internal surface within one or more lumen(s) 128 via, for example, an adhesive, a mechanical fit, a snap fit, a press fit, a weld, or any other coupling techniques commonly known in the art.

To help provide additional stability of medical device 102 relative to endoscope 104, one or more portions of medical device 102 may also be coupled to endoscope 104 elsewhere along the longitudinal length of endoscope 104. For example, a proximal portion of one or more sheath element(s) 117 and/or an intermediate portion of sheath element(s) 117 may be coupled to, for example, proximal portion 104A, and/or intermediate portion 104B, respectively, of endoscope 104. One or more portions of handle 106 may additionally or alternatively be coupled to one or more portions of endoscope 104, such as, for example at a handle (not shown) of endoscope 104.

Although not shown, endoscope 104 may include one or more grooves, channels, lumens, and/or other features to movably receive control element(s) 116 and/or sheath element(s) 117 of medical device 102. The one or more grooves, channels, lumens, and/or other features may at least partially extend internally (e.g., on an internal surface) or externally (e.g., on an external surface) along a longitudinal length of endoscope 104. For example, the one or more grooves may extend inward from an external surface of endoscope 104. Additionally or alternatively, medical system 100 could include an outer sheath (not shown) to at least partially enclose, protect, or otherwise encapsulate endoscope 104, sheath element(s) 117, and/or control element(s) 116.

Furthermore, although not shown, one or more portions of endoscope 104 (e.g., distal portion 104C) may be deflectable, for example, via one or more knobs, levers, buttons, or other controls on a proximal handle. In these aspects, distal portion 104C of endoscope 104 may be maneuvered while being delivered to the treatment site and/or positioned relative to the treatment site, for example, in a retroflex position, which may be used when the treatment site is in the subject's esophagus, stomach, duodenum, colon, or other portion of the GI tract. Accordingly, at least portions of medical device 102 may be flexible or semi-flexible, for example, in order to be maneuvered along with endoscope 104.

Control element(s) 116 and/or sheath element(s) 117 may extend externally to endoscope 104 (e.g., along an external surface of endoscope 104). In such an instance, end cap 108 may be fixedly or removably coupled to control element(s) 116 and/or sheath element(s) 117. For example, one or more portions of end cap 108 may be fixedly or removably coupled to control element(s) 116 and/or sheath element(s) 117 by techniques such as, for example, an adhesive, an overmold, a mechanical fit, a snap fit, a press fit, a friction fit, a weld, or any other techniques or fasteners commonly known in the art. Any combination of techniques for coupling end cap 108 to control element(s) 116 and/or sheath element(s) 117 is also contemplated (e.g., an adhesive and an overmold).

Referring to both FIGS. 1A and 1B, end cap 108 is coupled to endoscope 104, for example, at or near distal portion 104C of endoscope 104. Specifically, FIG. 1A illustrates end cap 108 in a first configuration, and FIG. 1B illustrates end cap 108 in a second configuration. In the first configuration, one or more materials (e.g., fluids, agents, powders, substances, etc.) may be enclosed or confined within a portion of end cap 108. In the second configuration, the one or more materials may be at least partially delivered, ejected, or released from end cap 108.

As shown in FIGS. 1A and 1B, end cap 108 includes a fixed, or outer, portion 132 and a movable, or inner, portion 134 contained or confined within fixed portion 132. Fixed portion 132 may be configured to, for example, help couple end cap 108 to distal portion 104C of endoscope 104. For example, end cap 108 may include a base 136 on a proximal end of fixed portion 132. Base 136 of fixed portion 132 may partially or completely surround an external surface of distal portion 104C of endoscope 104.

As shown in FIGS. 1A and 1B, base 136 may be positioned proximal to distal face 104D of endoscope 104. Base 136 may be coupled to the external surface of endoscope 104 by techniques such as, for example, an adhesive, a mechanical fit, a snap fit, a press fit, a friction fit, a weld, or any other techniques commonly known in the art. In some embodiments, base 136 may provide an anchor for end cap 108 to endoscope 104. Base 136 may be formed of a rigid or semi-rigid material (e.g. stainless steel, titanium, polypropylene plastic, high-density polyethylene plastic, acrylic, aluminum, brass, rubber, etc.). Base 136 may be sufficiently sized and shaped to receive, for example, distal portion 104C of endoscope 104.

Sheath element(s) 117 may be coupled to or otherwise abut a proximal face 136A of base 136. Control element(s) 116 may extend through base 136, for example, via one or more channels, openings, cutouts, etc. (not shown) extending longitudinally through respective portions of base 136. Control element(s) 116 may be coupled to a proximal face of movable portion 134.

Fixed portion 132 of end cap 108 may be configured to contain, or confine, one or more materials (e.g., one or more fluids, powders, substances, agents, etc.), for example, within a receptacle 138. Receptacle 138 may be defined by an inner wall 139A and an outer wall 139B. For example, receptacle 138 may be formed between an outward-facing (e.g., away from axis A) surface of inner wall 139A and an inward-facing (e.g., towards axis A) surface of outer wall 139B. In such a way, inner wall 139A and outer wall 139B may be concentric. A distal wall 139C may define a distal end of receptacle 138, and a distal face of movable portion 134 may define a proximal end of receptacle 138. In such a way, a volume of receptacle 138 may change as movable portion 134 is moved proximally or distally. For example, as movable portion 134 moves distally, the volume of receptacle 138 may decrease. Alternatively, as movable portion 134 moves proximally, the volume of receptacle 138 may increase.

The inward-facing surface of inner wall 139A may define an aperture 140 extending from a proximal end of end cap 108 to a distal end of end cap 108. For example, aperture 140 may be concentric with opening 137 of base 136 such that aperture 140 may be configured to receive a portion of distal portion 104C of endoscope 104 that is positioned within opening 137. For example, inner wall 139A may extend circumferentially around at least a portion of distal portion 104C of endoscope 104. In such a way, respective portions of distal portion 104C are within aperture 140 and opening 137. Furthermore, aperture 140 and/or opening 137 may span a full circumference of each respective portion of distal portion 104C. Although not shown, in some embodiments, aperture 140 and/or opening 137 may span less than a full circumference of each respective portion of distal portion 104C. Aperture 140 may have a same diameter and/or shape as opening 137 of base 136. Alternatively, aperture 140 may a different diameter (e.g., smaller or greater) and/or a different shape than opening 137 of base 136. For example, aperture 140 may be square and opening 137 may be circular or ovular, or vice versa.

The outward-facing surface of outer wall 139B may form an outer diameter of fixed portion 132. In some embodiments, the outer diameter of outer wall 139B may be the same or similar to an outer diameter of base 136. In alternative embodiments, the outer diameter of outer wall 139B may be greater or smaller than the outer diameter of base 136. A cross section of fixed portion 132 may be annular or ring-shaped.

Movable portion 134 may be contained, or confined, within fixed portion 132, for example, between inner wall 139A and outer wall 139B. Movable portion 134 may have an annular or ring shape and may be confined within, or disposed in, receptacle 138. Movable portion 134 may be configured to move axially (e.g., proximally and distally) within receptacle 138. Movable portion 134 may be formed from rigid and/or semi-rigid materials and configured to form a liquid-tight seal between the outward-facing surface of inner wall 139A and the inward-facing surface of outer wall 139B.

In some embodiments, movable portion 134 includes a proximal portion 134P and a distal portion 134D. For example, proximal portion 134P may be comprised of a rigid material (e.g., an acrylic material, a metallic material, etc.) for connection to control wire(s) 116 and abutting one or more biasing members 158 (discussed below). Distal portion 134D may be comprised of a semi-rigid or flexible material (e.g., a silicon, a rubber, polytetrafluoroethylene, nitrile, neoprene, ethylene propylene diene monomer rubber, fluorocarbon, etc.), for example to help maintain a liquid-tight seal within the respective portions of receptacle 138 and to also help push the material contained within receptacle 138 distally. In such a way, one or more fluids (e.g., one or more fluids, powders, substances, agents, etc.) may be contained within receptacle 138, distal to movable portion 134, and distal movement of movable portion 134 may urge the one or more materials distally.

As shown in FIGS. 1A and 1B, one or more support members 144 may extend between inner wall 139A and outer wall 139B, for example, across an entirety of a width of receptacle 138. In some embodiments, two support members 144 may form a wall configured to divide, or partition, receptacle 138 into a first section 138A and a second section 138B. In such a way, first section 138A and second section 138B may be partitioned by support members 144 such that first section 138A and second section 138B each has a defined volume. The two support members 144 may be evenly spaced, such that the volume of first section 138A and the volume of second section 138B are equal. In such an embodiment, an equal amount of material may be delivered from first section 138A and second section 138B, for example, as movable portion 134 moves distally. Alternatively, the two support members 144 may be unevenly spaced such that the volume of first section 138A and the volume of second section are unequal. For example, the volume of first section 138A may be greater than the volume of second section 138B, or vice versa. In such a way, different amounts of material may be delivered from each of first section 138A and second section 138B, for example, as movable portion 134 moves distally.

Additional support members 144 may further divide receptacle into additional equal or unequal sections. For example, three support members 144 may divide receptacle into three sections having equal or unequal volumes, four support members 144 may divide receptacle into four sections having equal or unequal volumes, etc. Accordingly, each section of receptacle 138 may be a same size, or each section of receptacle 138 may be a different size. For example, first section 138A may be larger than second section 138B, or vice versa.

Support members 144 may form a liquid-tight seal or wall between each of the sections. For example, support members 144 may form a liquid-tight seal or wall between first section 138A and second section 138B of receptacle 138. In such a way, a first material may be contained within first section 140A and a second material may contained within second section 140B, such that, for example, the first material and the second material do not mix, combine, or otherwise come in contact when within receptacle 138. Additional sections in receptacle 138 may contain additional materials.

In some embodiments, two or more support member(s) 144 may also divide movable portion 134 into two or more movable portions, such as, for example, a first movable portion 134A, a second movable portion 134B, etc. For example, some embodiments may include one, two, three, four, etc. movable portions 134. In such a way, each of the movable portions 134 (e.g., first movable portion 134A and second movable portion 134B) may be separated by two or more support member(s) 144 extending between inner wall 139A and outer wall 139B (e.g., substantially perpendicular to axis A).

In some embodiments, one or more support members 144 may extend from a proximal end of end cap 108 to a distal end of end cap 108, for example, parallel to axis A. In some embodiments, support member(s) 144 may extend at least partially into or through movable portion 134 such that movable portion 134 rides along support member(s) 144, similar to a rail or a track. Alternatively, support member(s) 144 may, at least partially, extend radially outward from inner wall 139A and/or radially inward from outer wall 139B. In further alternatives, support member(s) 144 may be fully detached from inner wall 139A and outer wall 139B. For example, support member(s) 144 may be a rod, a column, a post, etc. extending through movable portion 134, between base 136 and distal wall 139C. In such a way, support member(s) 144 may be configured to assist in preventing movable portion 134 from rotating within receptacle 138. Further yet, movable portion 134 may be divided or separated into first movable portion 134A, second movable portion 134B, etc. by two or more support members 144.

Each of the movable portions 134 (e.g., first movable portion 134A, second movable portion 134B, etc.) may be configured to move together or independently within a respective section (i.e., first section 138A and second section 138B) of receptacle 138. For example, first movable portion 134A may be configured to move within first section 138A of receptacle 138 and second movable portion 134B may be configured to move within second section 138B of receptacle 138. Additionally or alternatively, each of first movable portion 134A and second movable portion 134B may be independently movable from one another. For example, first movable portion 134A may be configured to move proximally and/or distally within first section 138A, independent from proximal and/or distal movement of second movable portion 134B within second section 138B, or vice versa. Movement of each movable portion 134 (e.g., first movable portion 134A, second movable portion 134B, etc.) may be controlled by respective portions of handle 106 and/or multiple handles 106.

In some embodiments, end cap 108 may further include, for example, one or more tubular members 146 extending distally from distal wall 139C of fixed portion 132. For example, in some embodiments, end cap 108 may include two tubular members 146. Each tubular member 146 may include an internal lumen 147. Internal lumen 147 may be in fluid communication (e.g., fluidly connected) with a respective section of receptacle 138 (e.g., first section 138A, second section 138B, etc.) via one or more openings 149 in distal wall 139C.

Additionally or alternatively, end cap 108 may include a nozzle 150. Nozzle 150 may be in fluid communication with internal lumen 147 of each tubular member 146. Accordingly, each internal lumen 147 may permit fluid communication between receptacle 138 and a nozzle 150, for example, via opening(s) 149 in distal wall 139C and nozzle 150. In some embodiments, a proximal end of nozzle 150 may be closed, for example, during insertion of medical system 100 to the treatment site. Once a desired position is reached, nozzle 150 may be opened, for example, by forcing the material(s) contained within end cap 108 distally. In such a way, nozzle 150 permits material(s) to flow distally and prevents material(s) from flowing proximally. the material(s) of end cap 108

The one or more tubular members 146 may be arranged to provide material flow between the one or more sections of receptacle 138 and nozzle 150. For example, when receptacle 138 is divided into two or more sections (e.g., first section 138A, second section 138B, etc.), two or more tubular members 146 may be utilized to enable material flow from each section of receptacle 138 to nozzle 150. At least one first tubular member 146 may be arranged on distal wall 139C (e.g., coupled to a respective opening149) to provide fluid communication with first section 138A of receptacle 138 and nozzle 150.

Additionally, at least one second tubular member 146 may be arranged on distal wall 139C (e.g., coupled to another opening 149) to provide fluid communication with second section 138B of receptacle 138 and nozzle 150. Additional tubular members 146 may be utilized for additional sections of receptacle 138. For example, although not shown, a third tubular member 146 may be arranged on distal wall 139C (e.g., couple to yet another opening) to provide material flow between a third section and nozzle 150. Furthermore, additional tubular members 146 may be utilized for each section (e.g., two tubular members 146 for first section 138A of receptacle), for example, to increase material flow from each section of receptacle 138 to nozzle 150. Alternatively or additionally, respective tubular members 146 may be different sizes (e.g., different lateral cross sectional widths) such that different internal lumens 147 may provide different material flow rates.

Each tubular member 146 may be formed from a rigid or semi-rigid material (e.g. polytetrafluoroethylene, expanded polytetrafluoroethylene, silicone, stainless steel, titanium, polypropylene plastic, high-density polyethylene plastic, acrylic, aluminum, brass, rubber, etc.). In some embodiments, each tubular member 146 may be a tubular member. The distal end of each tubular member 146 may curve inwards, for example, towards axis A such that the distal end of each tubular member 146 couples to an external surface of nozzle 150.

Nozzle 150 may be aligned with axis A, for example, concentric to aperture 140 of fixed cap portion 132. Nozzle 150 may be distal to (e.g., spaced distally to) distal wall 136C. Nozzle 150 may include a stepped tip, a tapered tip, or a luer lock tip. For example, a distal end of nozzle 150 may be tapered such that the tip can be cut at different intervals to adjust or otherwise control the amount of material being dispensed from end cap 108. Alternatively, nozzle 150 may be tapered such that a distal end of nozzle 150 has a smaller diameter than a proximal end of nozzle 150. Further still, nozzle 150 may include a luer lock to permit the attachment of needles or other attachments (not shown) to nozzle 150 (e.g., to a distal portion of nozzle 150). Furthermore, nozzle 150 may include a valve (e.g., a one-way valve, a duckbill valve, a check valve, etc.), for example, to permit material flow distally from nozzle 150 and prevent material flow proximally into nozzle 150.

In some embodiments, nozzle 150 may include a plurality of internal threads 152 extending from a proximal portion of nozzle 150 to a distal portion of nozzle 150. Internal threads 152 may be configured, for example, to help mix or combine the materials delivered from receptacle 138. For example, as movable portion 134 is moved distally, material contained within each section (e.g., first section 138A, second section 138B, etc.) of receptacle 138 is forced distally. Accordingly, the material from receptacle 138 is forced through tubular member(s) 146 and into nozzle 150 where the materials are mixed or combined, for example, with the help of the plurality of internal threads 152. Applying continuous force to the materials results in the material being delivered from nozzle 150, via a distal opening 154. For example, distal movement of movable portion 134 may result in materials being forced into nozzle 150 and distally out of distal opening 154.

In some embodiments, internal threads 152 may be fixed within a proximal and/or a distal portion of nozzle 150. Alternatively, internal threads 152 may be movable within nozzle 150. For example, internal threads 152 may be configured to rotate (e.g., counterclockwise or clockwise) about axis A. In such a way, as the material passes within nozzle 150, internal threads 152 are rotated within nozzle 150. Accordingly, the material may be mixed by internal threads 152.

Fixed portion 132, tubular member(s) 146, and/or nozzle 150 of end cap 108 may each be made from a clear, transparent, or semi-transparent material to permit visualization within end cap 108. Additionally or alternatively, end cap 108 may include one or more radiopaque markers or materials, for example, to permit visualization of end cap 108 while using ultrasound, x-ray, computerized tomography (CT), magnetic resonance imaging (MRI), fluoroscopy, etc. For example, one or more markers (not shown) may be arranged on or in end cap 108 to provide an indication of the state or status of end cap 108, amount of material delivered, the placement of end cap 108 within the subject, etc. The one or more markers may be used in addition to or in place of indicator(s) 124 on handle 106, described above. Additionally or alternatively, one or more components of end cap 108 (e.g. movable portion 134, nozzle 150, etc.) may be comprised of radiopaque materials (e.g., titanium, copper, strontium, silver, barium, etc.) to provide similar indications.

Referring still to FIGS. 1A and 1B, in some embodiments, one or more extensions 156 may be arranged around opening 137 of base 136, for example, on a distal face 136B of base 136. Extension(s) 156 correspond to the openings in base 136 through which control element(s) 116 extend. Additional extension(s) 156 may be arranged around opening 137 of base 136 that do not correspond to the openings in base 136 through which control element(s) 116 extend. For example, some embodiments may include two control element(s) 116 (as shown) and one, two three, four, etc. extension(s) 156. Two or more extensions 156 may be arranged equally or unequally around base 136. For example, two or more extensions 156 may be arranged on base 136 every 30 degrees, 60 degrees, 90 degrees, 180 degrees, etc. Alternatively, a first extension may be at 20 degrees, a second extension may be at 90 degrees, a third extension may be at 120 degrees, etc. Many other arrangements of extensions 156 are contemplated.

One or more biasing members 158 (e.g., springs, coils, etc.) may extend between a distal end of each extension 156 and the proximal face of movable portion 134. Additionally or alternatively, biasing member(s) 158 may extend between distal face 136B of base 136 and the proximal face of movable portion 134. For example, each biasing member 158 may extend distally within a space between inner wall 139A and outer wall 139B. Each biasing member 158 may be biased distally. For example, each biasing member 158 may be in a compressed state between extension(s) 156 and movable portion 134, or between base 136 and movable portion 134, such that the biasing member(s) 146 exert a distal force.

Biasing member(s) 158 may be in a first compressed state in the first configuration, for example, when movable portion 134 is in a proximal position within receptacle 138. As movable portion 134 is moved distally within receptacle 138 (i.e., when end cap 108 is in a second configuration), biasing member(s) 158 may transition to a second compressed state. The second compressed state of biasing member(s) 158 may be less compressed than the first compressed state of biasing member(s) 158 in the first configuration. For example, biasing member(s) 158 may have a first length in the first configuration of end cap 108 and a second length in the second configuration of end cap 108. The first length of biasing member(s) 158 in the first configuration (shown in FIG. 1A), may be shorter than the second length of biasing member(s) 158 in the second configuration (shown in FIG. 1B).

Some embodiments may include one, two, three, four, or more biasing member(s) 158. For example, some embodiments may include an equal number of extension(s) 156 and biasing member(s) 158 such that a biasing member 158 extends from each extension 156. Other embodiments may include an unequal number of extensions 156 and biasing members 158. For example, there may be a greater number of extensions 156 than biasing members 158 such that not every extension 156 includes biasing member 158. For example, some embodiments may include three extensions 156 and two biasing members 158. In further alternative embodiments, there may be a greater number of biasing members 158 than extensions 156. For example, some embodiments may include four biasing members 158 and two extensions 156. In such alternative embodiments, a proximal end of one or more biasing member(s) 158 may be coupled to distal face 136B of base 136 instead of the distal face of extension(s) 156.

A proximal end of each biasing member 158 may be fixed to the distal face of each extension 156 or to base 136 via, for example, an adhesive, a friction fit, a mechanical fit, a snap fit, a press fit, a weld, an overmold, or any other coupling techniques commonly known in the art. A distal end of each biasing member 158 may be coupled (e.g., directly or indirectly) to the proximal face of movable portion 134. For example, the distal end of each biasing member 158 may be fixed to the proximal face of movable portion 134 via an adhesive, a friction fit, a mechanical fit, a snap fit, a press fit, a weld, an overmold, or any other coupling techniques commonly known in the art.

Control element(s) 116 may extend through base 136 and/or extensions 156, for example, via one or more thru holes, channels, openings, lumens, cutouts, etc. Control element(s) 116 may be coupled to the proximal face of movable portion 134. In some embodiments, each of the one or more control elements 116 may extend through base 136 and through one or more extensions 156. Additionally or alternatively, control element(s) 116 may extend through base 136, extension(s) 156, and biasing member(s) 158. For example, control element(s) 116 may extend through a center of biasing member(s) 158.

As previously discussed, control element(s) 116 may be extended or retracted via handle 106. Specifically, first movable body 112 may be moved distally and/or proximally to extend and/or retract control element(s) 116. For example, as first movable body 112 is moved distally (e.g., by rotating knob 118 in the first direction), control element(s) 116 is/are extended and tension is decreased in control element(s) 116. As control element(s) 116 are extended, biasing member(s) 158 are decompressed, or lengthened, thereby exerting a distal force on movable portion 134. In such a way, movable portion 134 is pushed or urged distally within receptacle 138 by biasing member(s) 158, for example, to a second configuration, as shown in FIG. 1B. Accordingly, as movable portion 134 is moved distally within receptacle 138, the space (e.g., a volume) defining receptacle 138 decreases and material(s) contained within receptacle 138 are forced through tubular member(s) 146, into nozzle 150, and out of distal opening 154. In such a way, the material(s) once contained within receptacle 138 may be delivered to a treatment site.

Movable portion 134 may be moved distally within receptacle 138, for example, until a distal face of movable portion 134 abuts a proximal face of distal wall 139C. In such a way, movable portion 134 may transition from the first configuration shown in FIG. 1A to the second configuration shown in FIG. 1B. Movable portion 134 may transition from the first configuration to the second configuration, by extending or relaxing the tension of control element(s) 116. For example, as the user moves first movable body 112 distally by a first distance, control element(s) 116 move distally within sheath(s) 117 such that control element(s) 116 are extended or such that tension in control element(s) 116 is reduced. Reducing the tension in control element(s) 116 permits biasing member(s) 158 to expand. As biasing member(s) 158 expand, movable portion 134 is forced distally, thus decreasing the volume of receptacle 138. As the volume of receptacle 138 is decreased, material is forced distally into tubular member(s) 116, nozzle 150, and, ultimately, out of distal opening 154.

The transition from the first configuration to the second configuration may be gradual. For example, tension in control element(s) 116 may be gradually released such that movable portion 134 is gradually forced distally. For example, control element(s) 116 may have a first tension, a second tension less than the first tension, a third tension less than the first and second tensions, etc. In such a way, tension in control element(s) 116 may be decreased such that movable portion 134 moves distally to deliver approximately 20% of the material(s). Tension in control element(s) 116 may further decrease such that movable portion 134 moves distally an additional amount, for example, to deliver approximately 50% of the material(s). In such a way, tension in control element(s) 116 may be decreased such that movable portion 134 moves distally, for example, until the desired amount and/or approximately 100% of the material(s) is/are delivered. Indicator(s) 124 and/or other markings on handle 106 may indicate the amount of material delivered.

Movable portion 134 may be transitioned from the second configuration shown in FIG. 1B to the first configuration shown in FIG. 1A by, for example, retracting control element(s) 116 or increasing the tension of control element(s) 116. Control element(s) 116 may be retracted or tensioned by, for example, moving first movable body 112 proximally by a second distance (e.g., by rotating knob 118 in the second direction). As control element(s) 116 are retracted, movable portion 134 is pulled proximally within receptacle 138 by control element(s) 116. Biasing member(s) 158 may then be compressed, and medical device 102 may be returned to the configuration shown in FIG. 1A.

In some embodiments, material(s) may be loaded into end cap 108 via, for example, at least one port 160 arranged on a surface of fixed portion 132 of end cap 108. Additional ports 160 may be arranged on the surface of fixed portion 132, for example, to load different sections (e.g., first section 138A, second section 138B) of receptacle 138. For example, a first port 160 may be aligned with a portion of first section 138A and a second port (not shown) may be aligned with a portion of second section 138B such that a first material may be loaded into first section 138A via the first port 160 and a second material may be loaded into second section 138B via the second port (not shown). In such a way, two or more materials may be loaded into end cap 108. In some embodiments, port 160 may comprise a one-way valve. Material(s) may be loaded into end cap 108 via a delivery device such as, for example, a syringe.

In alternative embodiments, material(s) may be loaded into end cap 108, for example, when end cap 108 is outside of the subject and when movable portion 134 is pulled proximally. For example, as movable portion 134 is pulled proximally, a vacuum or otherwise reduced pressure is created within receptacle 138. Accordingly, material may be pulled or otherwise urged into end cap 108 via distal opening 154 on nozzle 150.

Material(s) may be loaded within different sections (e.g., first section 138A, second section 138B, etc.) of receptacle 138 by, for example, moving each movable portion (e.g., movable portion 134A, movable portion 134B) independently. For example, a first material may be loaded into first section 138A by pulling first movable portion 134A proximally, for example, to create a vacuum within first section 138A of receptacle 138. Similarly, a second material may be loaded into second section 138B by pulling second movable portion 134B proximally, for example, to create a vacuum within second section 138B of receptacle 138. Each movable portion 134 of end cap 108 may be moved by, for example, different portions of one or more handles 106.

A variety of materials may be delivered with medical device 102 (e.g., with end cap 108). As discussed above, in some embodiments, receptacle 138 may include two or more sections (e.g., first section 138A, second section 138B, etc.). In such a way, each section of receptacle 138 may allow for the materials within receptacle 138 to be kept separate until just before the materials are delivered to the treatment site. For example, a first material may include fibrinogen (e.g., lyophilized pooled human concentrate), and a second material may include thrombin (e.g., human or bovine thrombin). Accordingly, the first material and the second material are not combined until just before they are delivered. For example, the first and second material may be combined within nozzle 150. Immediately or shortly after being combine, the two materials may undergo a reaction (e.g., coagulation or other chemical reaction). Many other two-, three-, four-part materials may be utilized with end cap 108.

FIG. 2 is a flow diagram of an exemplary method 200 that one or more users may perform with any of the medical systems, devices, or portions of systems discussed herein, for example, medical system 100. In a first step 202 of method 200, medical system 100 (e.g., a distal portion of medical system 100) may be inserted into the subject, for example, through a natural orifice or incision. Medical system 100 may include, for example, medical device 102 and endoscope 104. Medical device 102 may further include handle 106 and end cap 108. First step 202 may also include coupling end cap 108 to distal portion 104C of endoscope 104 prior to inserting medical system 100 into the subject. First step 202 may further include deflecting and/or positioning distal portion 104C of endoscope 104 (e.g., via knobs, levers, dials, etc.) to be adjacent to or otherwise positioned relative to the treatment site.

Once medical system 100 is in the desired position, a second step 204 includes moving movable portion 134 of end cap 108 distally within receptacle 138, for example, by manipulating first movable body 112 of handle 106 relative to stationary body 114 (e.g., distally relative to stationary body 114). Moving first movable body 112 of handle 106 relative to stationary body 114 may relieve or decrease tension in control element(s) 116. As tension in control element(s) is decreased, biasing member(s) 158 may expand and push or otherwise urge movable portion 134 distally relative to fixed portion 132. Movement of movable portion 134 distally decreases the volume of receptacle 138, such that at least some material contained within receptacle 138 is delivered to the treatment site, for example, via nozzle 150. Indicator(s) 124 on handle 106 may provide an indication for an approximate amount of material delivered.

In some embodiments (e.g., embodiments with two or more materials separately contained within end cap 108), the two or more materials may be combined or mixed within nozzle 150. Once combined within nozzle 150, the two materials may be combined and/or activated (e.g., undergo a reaction). The combined material may then be delivered to the treatment site, for example, via nozzle 150.

In an optional step 206 of method 200, for example, after at least some material is delivered from end cap 108, the user(s) may reposition end cap 108 within the subject. For example, the user(s) may move endoscope 104 proximally or distally within the subject. Optional step 206 may also include deflecting and/or positioning distal portion 104C of endoscope 104 (e.g., via knobs, levers, dials, etc.). Once end cap 108 has been repositioned, the user(s) may deliver more of the material(s), as described above.

In a step 208, for example, once a desired amount of material is delivered to the treatment site, the user(s) may remove medical system 100 from the subject. For example, medical system 100 may be removed by retracting endoscope 104 proximally through the natural orifice or incision. Once medical system 100 is removed, method 200 may be repeated, for example, with a second end cap 108 or the same end cap 108 with new or additional material(s) loaded, for example, into one or more sections of receptacle 138.

It will be apparent to those skilled in the art that various modifications and variations can be made to the disclosed device without departing from the scope of the disclosure. Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

We claim:

1. A medical device, comprising:
a handle, wherein the handle includes a first movable body and a stationary body;
an end cap, the end cap comprising:
an outer portion;
an inner portion disposed within the outer portion; and
at least one biasing member,
wherein the outer portion of the end cap defines a receptacle configured to contain a material, wherein the inner portion is proximal to the receptacle within the outer portion, wherein the inner portion is coupled to the first movable body by at least one control element, wherein the at least one biasing member is disposed between a proximal face of the inner portion and a distal face of a proximal wall of the outer portion such that movement of the first movable body relative to the stationary body moves the inner portion of the end cap relative to the outer portion of the end cap, and wherein movement of the inner portion of the end cap is configured to deliver the material.

2. The medical device of claim 1, wherein, in a first configuration, (a) the at least one control element has a first tension, (b) the at least one biasing member has a first length, and (c) the inner portion is in a first position; and
wherein, in a second configuration, (d) the at least one biasing member has a second length greater than the first length, (e) the at least one biasing member has a second length greater than the first length, and (f) the inner portion is in a second position.

3. The medical device of claim 2, wherein, in the first configuration, the receptacle has a first volume, and wherein, in the second configuration, the receptacle has a second volume, wherein the second volume is less than the first volume.

4. The medical device of claim 1, wherein the end cap further comprises:
a nozzle, and
at least one tubular member extending distally from a distalmost face of the outer portion, wherein the at least one tubular member is fluidly connected to the nozzle and the outer portion, and wherein the material is delivered via the at least one tubular member and the nozzle.

5. The medical device of claim 4, wherein the nozzle includes a plurality of internal threads.

6. The medical device of claim 1, wherein the receptacle includes at least a first section and a second section, wherein at least the first section and the second section are partitioned by a wall, wherein the material is a first material, and
wherein the first section is configured to contain the first material, and the second section is configured to contain a second material.

7. The medical device of claim 6, wherein the first section of the receptacle has a first volume, and wherein the second section of the receptacle has a second volume different from the first volume.

8. The medical device of claim 1, wherein the end cap includes at least one port arranged on an external surface of the outer portion.

9. The medical device of claim 8, wherein the at least one port includes a one-way valve.

10. The medical device of claim 1, wherein the at least one control element is moveably disposed within a lumen of a sheath.

11. The medical device of claim 10, wherein the sheath is coupled to the outer portion of the end cap.

12. The medical device of claim 1, wherein the end cap is removably coupled to an endoscope by one or more fasteners, adhesives, or a friction fit.

13. The medical device of claim 1, wherein the end cap includes an aperture extending from a proximal end to a distal end, wherein the aperture is configured to receive a distal portion of a scope.

14. The medical device of claim 1, wherein the inner portion includes a proximal portion and a distal portion, wherein the proximal portion is a rigid material, wherein the distal portion is a flexible material, and wherein the distal portion of the inner portion forms a liquid-tight seal with the outer portion of the end cap.

15. The medical device of claim 1, wherein the handle includes at least one indicator, wherein the at least one indicator indicates a volume of the material disposed within the receptacle of the end cap.

16. A medical device, comprising:
an end cap, the end cap comprising:
a fixed portion;
a first movable portion; and
a second movable portion,
wherein the fixed portion of the end cap includes a first section and a second section partitioned by a wall, wherein the first section and the second section are configured to receive first and second materials, respectively,
wherein the first movable portion is disposed within the first section and the second movable portion is disposed within the second section;
wherein, in a first configuration, the first movable portion is in a proximal position within the first section of the fixed portion, and the second movable portion is in a proximal position within the second section of the fixed portion, wherein each of the first section and the second section has a first volume; and
wherein, in a second configuration, the first movable portion is in a distal position within the first section of the fixed portion such that the first section has a second volume, wherein the second volume is less than the first volume.

17. The medical device of claim 16, wherein, in the second configuration, the second movable portion is in a distal position within the second section of the fixed portion such that the second section has a second volume, wherein the second volume is less than the first volume.

18. The medical device of claim 16, wherein the end cap includes at least two biasing members, wherein a first of the at least two biasing members is disposed between a proximal face of the first movable portion and a distal face of a proximal wall of the fixed portion, wherein a second of the at least two biasing members is disposed between a proximal face of the second movable portion and the distal face of the proximal wall of the fixed portion;
wherein, in the first configuration, each of the at least two biasing members has a first length; and
wherein, in the second configuration, the first of the at least two biasing members has a second length, wherein the second length is greater than the first length.

19. A method, comprising:
delivering a medical system to a treatment site, wherein the medical system includes:
a scope;
a medical device, the medical device comprising:
a handle having a first movable body and a stationary body;
an end cap removably coupled to a distal end of the scope, the end cap comprising an inner portion, an outer portion, and at least one biasing member, wherein the inner portion is coupled to the first movable body of the handle by one or more control elements, wherein the outer portion defines a receptacle, wherein the at least one biasing member is disposed between a proximal face of the inner portion and a distal face of a proximal wall of the outer portion; and
a material, wherein the material is disposed within the receptacle of the end cap;
moving the first movable body relative to the stationary body by a first distance such that (a) a tension in the one or more control elements is decreased by a first amount and (b) the at least one biasing member is extended by a first amount to move the inner portion of the end cap relative to the outer portion of the end cap by a first distance; and
delivering a first amount of the material.

20. The method of claim 19, further comprising:
repositioning the medical system relative to the treatment site;
moving the first movable body relative to the stationary body by a second distance such that (a) a tension in the one or more control elements is decreased by a second amount and (b) the at least one biasing member is extended by a second amount to move the inner portion of the end cap relative to the outer portion of the end cap by a second distance; and
delivering a second amount of the material.
